(19) United States
(12) Patent Application Publication
(10) Pub. No.: US 2006/0126019 A1
(43) Pub. Date: Jun. 15, 2006

(54) Methods and systems for wavefront analysis

(76) Inventors: Junzhong Liang, Fremont, CA (US); Dawei Zhu, San Jose, CA (US)

Correspondence Address: XIN WEN, 3449 RAMBOW DRIVE, PALO ALTO, CA, 94306, US

(21) Appl. No.: 11/293,612

(22) Filed: Dec. 2, 2005

Related U.S. Application Data
(60) Provisional application No. 60/635,247, filed on Dec. 10, 2004.

(51) Int. Cl. A61B 3/00

(52) U.S. Cl. 351/246

(57) ABSTRACT

A method for analyzing wavefront sensing images as an array of focus spots comprise obtaining a wavefront sensing image of an optical object such as an eye using a Hartmann-Shack wavefront sensor, determining at least one average distance between the neighboring focus spots in the wavefront images. The method for analyzing wavefront sensing images further includes calculating a sphero-cylindrical error, detecting the focus spots of the wavefront image automatically, calculating the wavefront slopes at an array of sampling locations, and reconstructing the wave aberration of the tested optical object from the measured wavefront slopes using a least-squares estimator. The least-squares estimator includes a modal wavefront reconstruction using Zernike polynomials with a Zernike order larger than 10 and less than or equal to the number of sampling points along one axis in the sampled area. The least-squares estimator also includes a mixed modal-zonal least-squares estimation by extracting a set of wavefront modes using a least-squares modal wavefront estimator, calculating the residual wavefront slopes at the sampling position of the wavefront sensor, reconstructing a residual wavefront using a least-squares zonal estimator, and obtaining the wave aberration of the tested optical object by combining the wavefront modes from the modal estimator and the reconstructed residual aberrations from the zonal estimator.

### CROSS-REFERENCES TO RELATED INVENTIONS

The present invention claims priority to the provisional U.S. patent application 60/635,247, titled “Algorithm and methods for wavefront analysis” filed on Dec. 10, 2004 by Liang et al. The present invention is related to commonly assigned and concurrently filed U.S. patent application “Improved methods and apparatus for wavefront sensing of human eyes” filed by Liang. The disclosures of these related applications are incorporated herein by reference.

### TECHNICAL FIELD

This application relates to systems and methods for measuring optical defects using wavefront sensing technologies, in particular, for measuring aberrations of human eyes.

### BACKGROUND

Wavefront-guided vision correction is becoming a new frontier for vision and ophthalmology. It offers supernormal vision beyond conventional sphero-cylindrical correction, allowing the imaging of living photoreceptors and the perfection of laser vision correction. Wavefront technology will reshape the eye care industry by enabling customized design of laser vision correction, contact lenses, intro-ocular lenses, and even spectacles. The first precise method for the detection of wave aberrations was disclosed in “Objective measurement of wave aberrations of the human eye with the use of a Hartmann-Shack wave-front sensor” J. Opt. Soc. Am. A, vol. 11, no. 7, p. 1949, by Liang et al., in July, 1994.

Wavefront sensors for the eye using a Hartmann-Shack sensor are usually designed with a dynamic correction of eye's focus error (myopia and hyperopia) using a trombone optical system and the correction of astigmatism using cylindrical lenses. Correcting for the sphero-cylindrical corrections in the eye significantly reduces demands for the wavefront sensor so that the sensor only measures the high-order aberrations in the eye. However, correcting for the sphero-cylindrical refractive error in the eye also has a few disadvantages. First, it often involves in adding significant cost. Second, it can introduce unwanted high-order aberrations when the correction is not perfect. Third, it prolongs wavefront measurements because the sphero-cylindrical correction in the tested eye must be first determined and then corrected by moving optical components. Moving components increases complexity in a wavefront system and can cause problems for long-term stability and reliability.

Focus error or the spherical correction (myopia or hyperopia) is the largest refractive error in the eye. For the vast majority of the population that needs vision correction, the spherical correction is in the range between −12D (myopia) and +6D (hyperopia). If the sphero-cylindrical errors are not corrected in a wavefront sensor with a Hartmann-Shack sensor, focus spots of lenslets can be moved far beyond the physical boundaries set by the lenslets. This causes difficulties for an automatic spot identification. A clear need exists in the art to develop practical algorithms for the automatic identification of focus spots for wavefront measurements of human eyes without a conventional sphero-cylindrical correction. Flexibility in dynamic spot finding can lead to cost-effective wavefront refractors with improved accuracy and reliability.

Wave aberration of human eye measured from wavefront refractors are normally reconstructed using truncated Zernike polynomials. Zernike polynomials up to fourth order were first chosen to include all primary Seidel aberrations (Liang '94). Later, Zernike polynomials up to the 10^{th} order were selected to include more aberrations for the description of normal eye's wave aberration (Liang '98). The selection of Zernike aberrations up to the 10^{th} order for the wavefront reconstruction has been based on the understanding of possible aberrations in normal human eyes, not based on aberrations in all eyes including those with abnormal vision or after complicated refractive surgeries. In order to best represent the optical defects in all possible eyes, a need exists in the art to develop an improved algorithm for wavefront reconstruction that include Zernike aberrations limited by the measured wavefront slopes only, not by a fixed and pre-determined Zernike order.

Wavefront reconstruction involves a least-squares estimation of the wavefront function from measured wavefront slopes at an array of sampling positions. The least-squares estimations are usually conducted in one of the two approaches (Southwell, 1980): a) Modal wavefront estimators that use a set of polynomials with their coefficients of polynomials to be determined by a least-squares estimation, and b) Zonal wavefront estimators that determines the wavefront values at the sampling points from a least-squares estimation with the measured wavefront slopes. Modal wavefront reconstruction is best suited for fitting wavefronts that are dominated by a few specific aberration modes. It extracts the wavefront modes without being limited by limited sampling and filters out the unwanted noises. It is, however, limited for fitting localized features that are not well characterized by the selected wavefront modes. Zonal wavefront reconstruction is best suited for fitting localized aberrations, but is limited when a few particular modes dominate the wavefront shape. Aberrations in human eyes have two important features. First, aberrations in human eyes can be dominated by a few global aberrations such as the focus error, astigmatism, coma, spherical aberrations. Second, aberration in surgical eyes and abnormal eyes may contain significant irregular aberrations that are localized in nature. This poses a challenge to use either Modal-based or Zonal-based wavefront reconstruction. A need exists in the art to develop a mixed modal-zonal wavefront estimators that takes advantages of both modal and zonal wavefront estimators for the best estimation of wave aberration in the eye.

### SUMMARY

Implementations of the system may include one or more of the following. In one aspect, the present invention relates to a method for analyzing wavefront sensing images of a Hartmann-Shack wavefront sensor, comprising:

obtaining a wavefront sensing image of an optical object having at least one optical surface using the Hartmann-Shack wavefront sensor, wherein the wave sensing image comprises an array of focus spots; and

determining at least one average distance between the neighboring focus spots in the wavefront images.

In another aspect, the present invention relates to a method for characterizing the wave aberration of an optical object having at least an optical surface, comprising:

measuring wavefront slopes at an array of sampling points; and

reconstructing the wave aberration of the optical object using the wavefront slopes and a set of Zernike polynomials, wherein the Zernike order is dynamically set to be larger than 10 and less than or equal to the total number of sampling points along one axis in the sample area.

In yet another aspect, the present invention relates to a method for characterizing the wavefront of an optical object having at least an optical surface, comprising:

measuring wavefront slopes at an array of sampling points;

using a modal wavefront estimator to extract a set of wavefront modes from the measured wavefront slopes;

subtracting the set of wavefront modes from the measured wavefront slopes to obtain residual wavefront slopes at the sampling points of the wavefront sensor;

using a zonal wavefront estimator to reconstruct the residual aberrations from the residue wavefront slopes; and

determining the wave aberration of the optical object by combining the set of wavefront modes and the residual aberrations.

Embodiments may include one or more of the following advantages. The invention system and methods provide an algorithm to determine the average distance between the neighboring focus spots in two perpendicular directions without finding any focus spot in a wavefront image of a Hartmann-Shack sensor. The average distances of focus spots allow automatic detection of the defocus (the correction for myopia and hyperopia) and astigmatism (the cylindrical correction) in the tested eye. The invented algorithm enables a design of a conventional auto-refractor using a Hartmann-Shacks sensor without the complicated process in finding the focus spots of the lenslet array. It also enables automatic detection of focus spots of a Hartmann-Shack sensor without the need for dynamic correction of eye's sphero-cylindrical correction. Knowing the average spot distance (defocus and astigmatism in the eye) allows a dynamic boundary to be set up around each focus spot as if the defocus and astigmatism in the eye are physically corrected in the measurements, and thus enable to measure wave aberration for the eye without the correction of eye's focus error and astigmatism.

Another advantage of the invention system and methods is that they improve the wavefront reconstruction from wavefront slopes. An improved least-squares wavefront estimator is developed to include Zernike polynomials limited by the measured wavefront slopes instead of by pre-determined Zernike polynomials known in the prior art. A mixed Modal-Zonal wavefront estimator is developed capable of extracting a set of dominating wavefront modes from the measured wavefront slopes as well as fitting the irregular aberrations without the limitations of a modal wavefront estimator.

Yet another advantage of the invention system and methods is that the optical defects in eyes are more accurately represented by high-order Zernike polynomials for a given wavefront sensor. The disclosed techniques are applicable to those eyes with abnormal vision and eyes after refractive surgeries, which overcomes a major difficulty facing the present vision correction technologies.

Although designed for measuring wave aberration of an eye, the methods and systems in present invention can also be applied to a wavefront measurement of any optical object having at least one optical surface including ophthalmic lenses.

The details of one or more embodiments are set forth in the accompanying drawings and in the description below. Other features, objects, and advantages of the invention will become apparent from the description and drawings, and from the claims.

### DRAWING DESCRIPTIONS

FIG. 1 shows a schematic diagram of a wavefront sensing system in accordance with the present invention.

FIGS. 2a and 2b shows wavefront-sensor images captured by a Hartmann-Shack sensor for a hyperopic eye and a myopic eye, respectively.

FIGS. 2c and 2d show the one-dimensional profiles respectively for the images in FIGS. 2a and 2b.

FIGS. 2e and 2f are the Fourier spectra of the one-dimensional profiles shown in FIGS. 2c and 2d respectively.

FIG. 3 shows a block diagram for the method of dynamic spot finding in the wavefront analysis in accordance with the present invention.

FIG. 4 is a plot showing the relationship between the number of Zernike modes (N) and Zernike order (n) and the relationship between the total number of wavefront slopes (M) and the number of sampling points (m) across one axis in a circular pupil.

FIG. 5 shows the condition number of the pseudo-inverse matrix used in modal wavefront estimation with Zernike polynomials, where n is the order of Zernike polynomials and m is the number of sampling points alone one axis in a circular pupil.

FIG. 6 shows the flow diagram for a mixed zonal-modal wavefront estimator for wavefront reconstruction from wavefront slopes.

### DETAILED DESCRIPTION

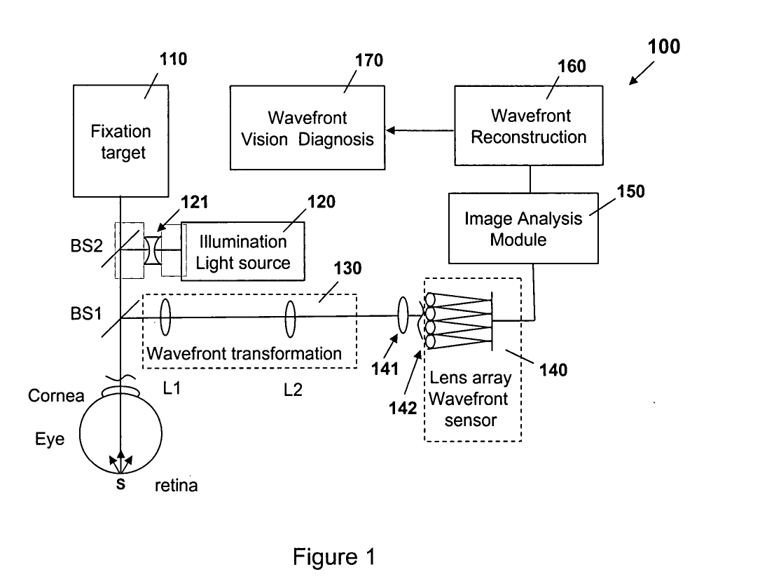

FIG. 1 illustrates a schematic diagram of a wavefront-sensing system 100 in accordance with the present invention. A fixation system 110 is provided to stabilize the tested eye for accommodation control (i.e. the control of the eye's focus position). A collimated light source 120 is converted to a small divergent beam of light by a negative spherical lens 121. The divergent beam is reflected off a beam splitter (BS2) and generates a compact light source (S) at the retina of the eye. The compact light beam illuminates the eye's retina and is diffusely reflected by the retina. The compact light beam can be referred as the probing light beam or the illumination light beam. The reflected light propagates to the eye's cornea and forms a distorted wavefront at the cornea plane. The distorted wavefront is reflected off a beam splitter BS1 and then relayed by an optical relay system 130 to a Hartmann-Shack wavefront sensor 140. The optical relay system 130 consists of lenses (L1) and (L2). A cylindrical lens 141 introduces a fixed cylindrical wave to the wavefront from the eye before it enters a Hartmann-Shack wavefront sensor 140. The Hartmann-Shack wavefront sensor 140 includes a lenslet array and an image sensor. The lenslet array 142 converts the distorted wavefront to an array of focus spots on the image sensor. An image analysis module 150 detects the focus spots and calculates the slopes of the wavefront. A wavefront estimator 160 reconstructs the wavefront using the slopes of the wavefront. A vision diagnosis module 170 determines the eye's optical quality and optical defects, which can provide the basis for a vision correction diagnosis.

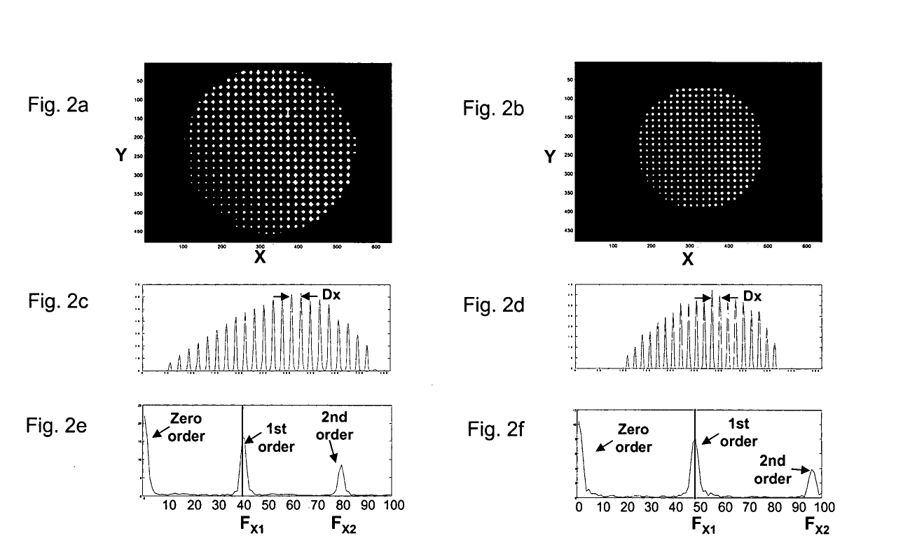

FIGS. 2a and 2b shows the wavefront sensor image captured by a Hartmann-Shack sensor for a hyperopic eye and myopic eye, respectively. The distance between neighboring focus spots is larger for a hyperopic eye corresponding to FIG. 2a, and smaller for a myopic eye corresponding to FIG. 2b. In both cases, most focus spots are beyond the physical boundaries of the lenslet array 142. The average distance between neighboring focus spots relates directly to the focus error of the tested eye. If the tested eye has astigmatism, the distance between neighboring focus spots in x- and y-directions will be different. Their difference relates directly to the astigmatism of the tested eye. Under the circumstances when the focus error and the astigmatism are not physically corrected, the challenge for automatic identification of the focus spots is the ability to determine the defocus and astigmatism of the tested eye without finding one focus spot in the wavefront sensor image.

The invention system and methods provide two approaches for finding the average spot distance in x- and y-directions without finding focus spots in a wavefront image. In the first approach, two-dimensional wavefront sensor images in FIGS. 2a and 2b are respectively converted to one-dimensional intensity profiles by averaging the intensity profile alone x- and y-axis as shown in FIGS. 2c and 2d. (For simplicity, only data in the horizontal axis are shown.) The distance between neighboring spots in the two-dimensional (2D) image can be determined by finding the distance between neighboring peaks in the one-dimensional (1D) profiles. The second approach uses the reciprocal relationship between the spot distance in the image domain and the spatial frequency in the Fourier domain as shown in FIGS. 2e and 2f. These two approaches can be used independently or jointly.

FIGS. 2c and 2d show the 1D profiles respectively for the images in FIGS. 2a and 2b. The 1D profiles along the X-axis are obtained by averaging the wavefront images along the y-direction. Because the lenslets 142 are distributed a 2D periodic pattern, the intensity profiles of the focus spots are nearly periodic. When an emmotropic eye is measured, the average distance between the neighboring peaks “Dx” equals to the spacing between lenslets. Dx is larger for the hyperopic eye (FIG. 2c) and smaller for the myopic eye (FIG. 2d). The average distance between the peaks from the 1D profiles can be calculated, which gives the distance between the neighboring spots in the x-direction in the wavefront images in FIGS. 2a and 2b.

Another way to determine the distance between peaks in a nearly periodic signal is to use Fourier analysis. The Fourier transforms of the 1D profiles in FIGS. 2c and 2d are respectively shown in FIGS. 2e and 2f. The Fourier transforms include a zero order, a first order, and a second order spectrum peaks. The spatial frequencies (Fₓ₁) of the spectrum peaks at different orders can be determined. The average distance between the neighboring peaks (Dx) can be obtained using the reciprocal relationship
Dx=c/Fₓ₁.
where c is a constant that can be determined analytically or empirically.

The average spot distances in x- and y-direction can be used to determine the refraction power of the eye in x- and y-direction, from which the sphero-cylindrical power of the eye can be determined. The Fourier analysis of wavefront images allows to determine the conventional sphero-cylindrical corrections without an need to identify a single focus spot in the wavefront image. It enables to design a cost-effective conventional auto-refractor or a lensometer for measuring ophthalmic lenses using a Hartmann-Shack wavefront sensor without a digital computer.

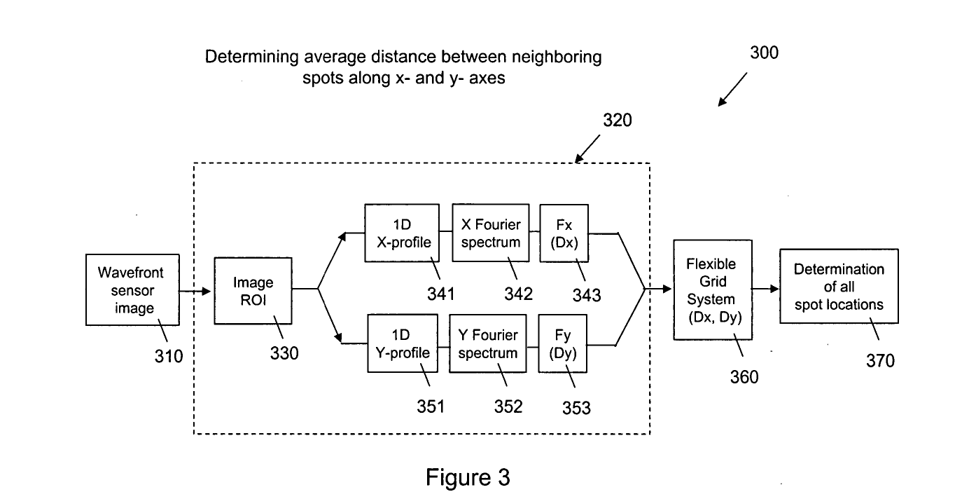

Another embodiment of the present inventions is to design a wavefront sensing system for the determination of the wave aberration of the eye including high order aberrations such as coma, spherical aberration without the need to correct for the sphero-cylindrical aberrations. FIG. 3 shows a block diagram 300 for the method of dynamic spot finding in the wavefront analysis in accordance with the present invention. A wavefront sensor image is first obtained in step 310. The average distance between the neighboring focus spots are determined in a series of steps in process 320. A region of interest (ROI) in the wavefront sensor image is selected in step 330. 1D profiles are obtained along the x- and the y-directions in steps 341 and 351 respectively. Their corresponding Fourier spectra are next calculated in steps 342 and 352. Finally, the spatial frequencies Fx and Fy of the first order peaks are determined. The average spot distance (Dx and Dy) is calculated in steps 343 and 353. Focus spots of wavefront images can be identified in step 360 with a flexible grid system based on the average spot distance in x- and y-directions.

A preferred embodiment for the automatic spot finding consists of the following steps. First, the average intensity of the wavefront image (Iₐᵥₑ) is determined. The original image is converted to a new image by keeping the intensity unchanged if the intensity at the pixel is above the averaged intensity, and by making the intensity zero if the intensity at the pixel is equal to or less than the averaged intensity. Such a pre-processing process can remove noise in the image. Second, the centroid of the pre-processed image is determined. The center of the wavefront measurement should be around the centroid of the image. Third, we find the first focus spots around the center of the wavefront image. The average distance in x- and y-direction enables us to setup a square (or rectangular) region (R1) around the centroid of the image within which there is one focus spot. Within the defined region (R1) we can find the pixel (P) with the peak intensity. The center of the focus spot should be close to the pixel P. Around the pixel with peak intensity we can define a new region (R2) that is about half the size of the averaged spot distance, and find the exact position of the focus spot by calculating the centroid of the intensity within R2. Fourth, we can find the neighboring four focus spots (top, bottom, left and right) around the first identified spot if the lenslets form a square array. From the average spot distances in x- and y-directions, we can find rough coordinates of these focus spots. Around the rough coordinates (x, y), we define a square (or rectangular) region R1 again with which there is one and only one focus spot. Within the defined region R1 we can find the pixel (P) with the peak intensity. The center of the focus spot should be close to the pixel P. Around the pixel with the peak intensity we define a new region R2 again that is about half the size of the averaged spot distance, and find the exact position of the focus spot by calculating the centroid of the intensity within R2. Using the same process we can automatically detect all focus spots in the wavefront image. Wavefront slopes in the measurement are determined from the displacements of each focus spot in referenced to the pre-determined coordinates when an aberration-free measurement is measured. Wavefront aberration of the tested eye can be reconstructed from the measured wavefront slopes from a wavefront sensor.

Zernike polynomials are often used for the representation of eye's wave aberration because Zernike polynomials form a complete orthogonal set over a circular area. The classic Seidel aberrations in optical systems are represented by just a few Zernike modes. Early studies of normal human eyes for a large pupil (7.3 mm) have shown that the magnitude of Zernike aberrations decreases monotonically as the Zernike order increases and Zernike aberrations beyond the 8^{th} order are negligible for normal human eyes. It was concluded truncated Zernike polynomial up to 10^{th} order were sufficient to represent eye's wave aberration. Selection of a fixed Zernike polynomials up to the 10^{th} order may be appropriate for representing wave aberration in normal human eyes, but not suitable for all eyes including those with abnormal vision or those after complicated refractive surgeries,

It is important to determine all aberrations based on the measured wavefront slopes beyond a fixed Zernike polynomials up to the 10^{th} order.

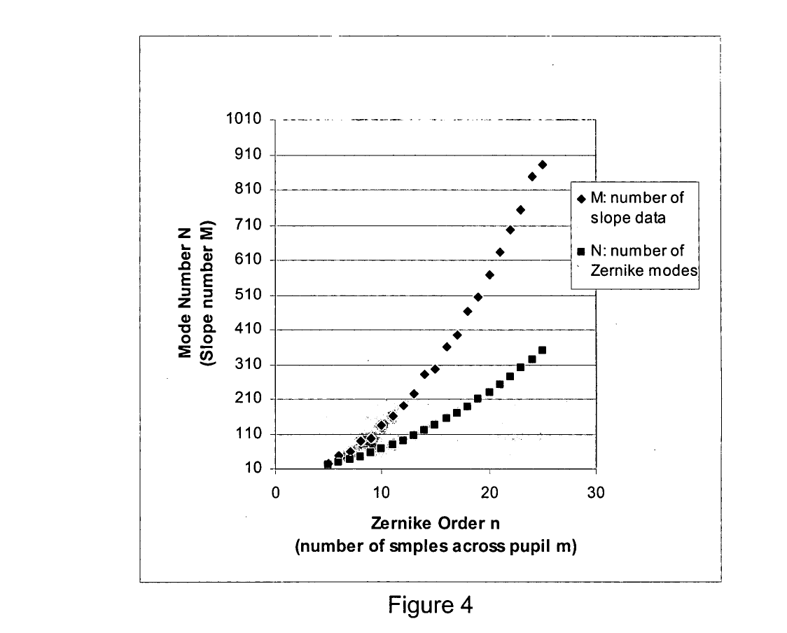

Wavefront reconstruction using Zernike polynomials involves in solving linear equations with a number of unknown variables (i.e. the Zernike coefficients) from a set of linear equations provided by the slope data. For a circular pupil, the total number “M” of wavefront data (including both x- and y-slopes) within the circular pupil is approximately
M=0.5*pi*(m−1)², (1)
where “m” is the number of sampling points across the pupil along one axis. On the other hand, the total number “N” of the Zernike modes for a Zernike polynomial truncated to the nth order can be expressed as
N=0.5*(n²+3n). (2)
The wavefront reconstruction involves solving linear equations with “N” unknown variables (the Zernike coefficients) from “M” linear equations. The functional relationship between “M” and “m” and between “N” and “n” in Equations (1) and (2) are illustrated in FIG. 4.

A least-squares solution of the linear equations requires the number of measurement (M) should be more than the number of Zernike mode N so that a least-square estimation of Zernike coefficients can be found using a pseudo-inverse matrix. In the early studies known in the prior art, low-order Zernike polynomials with a small number of Zernike modes are often used to ensure the number of measurement is far more than the number of Zernike modes. It is, however, not known in the prior art, for a given set of wavefront slopes, what is the highest order Zernike polynomials allowed for a wavefront reconstruction.

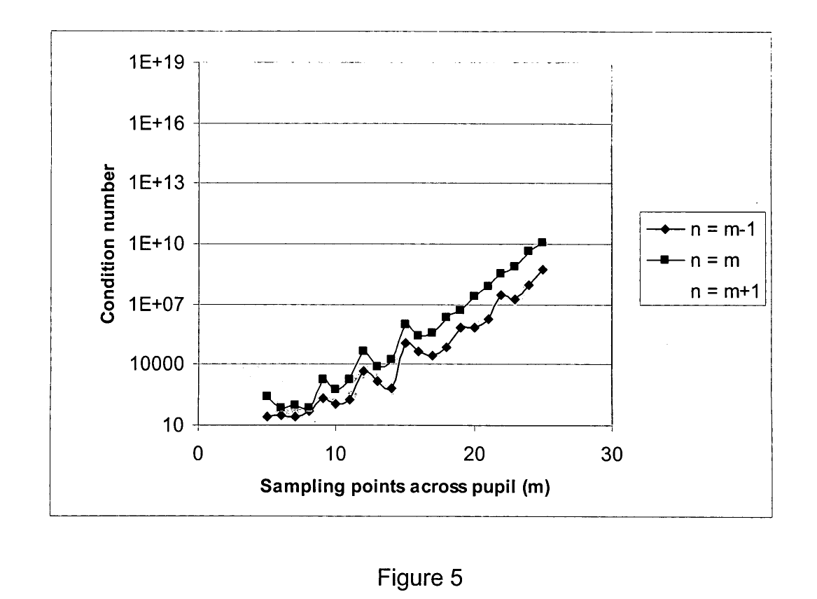

Simulations were conducted to determine conditions for stable solutions using the largest number of Zernike order (n) for a given number of sampling point (m) across a circular pupil. We found that an unstable solution occurs if “n” is larger than “m”, which manifests itself in extremely larger condition number for the pseudo-inverse matrix as shown in FIG. 5. Conditional number is the ratio of the largest to smallest singular value in the singular value decomposition of a matrix. Condition numbers were found to be extremely large and in a similar range (>10¹⁷) when Zernike order (n) is larger than the number (m) of sampling point across the pupil by 1, which resulted in an unstable least-squares solution and erroneous wavefront in our simulation.

When the Zernike order (n) is equal to or less than the number (m) of sampling points across the pupil, the condition numbers were found to be several magnitudes smaller. Simulation results also showed negligible wavefront errors when the Zernike order is equal to or smaller than the number of sampling data across the pupil.

Simulations using known Zernike coefficients and calculated wavefront slopes confirmed that significant reconstruction errors occur when the Zernike order (n) is larger than the number of sampling points (m) across a circular pupil even thought the pseudo-inverse is still over-determined. Error is negligible if the Zernike order (n) is less than or equal to the number of wavefront samples across a circular pupil (m).

We conclude that the highest order Zernike polynomials (n) allowed for wavefront reconstruction from wavefront slopes has to be equal to or less than the number of sampling points (m) across a circular pupil. Using this criterion, we can reliably estimate Zernike aberrations beyond 10^{th} order if the sampling point across the tested pupil is more than 10, and thus improve accuracy in representing wavefront measurement form the eye.

Wavefront reconstruction 160 is a critical step of the wavefront measurement. Wavefront estimators in the prior art fall into two fundamental categories: zonal wavefront estimation and modal wavefront estimation. Aberrations in human eyes have two important features. First, aberrations in human eyes can be dominated by a few global aberrations such as the focus error, astigmatism, coma, spherical aberrations. Second, aberration in surgical eyes and abnormal eyes may contain significant localized irregular aberrations. Neither of the two approaches is effectively for handling both features of aberrations in the human eyes. Modal wavefront reconstruction is best suited for fitting wavefronts that are dominated by a few specific aberration modes. It extracts the wavefront modes without the need for a large number of sampling points but filters out unwanted noises as well as localized features that are not well represented by the Zernike modes. On the other hand, zonal wavefront estimator is best suited for fitting localized aberrations, but is not effective when a few particular Zernike modes dominate the wavefront shape. Moreover, zonal wavefront reconstruction is more sensitive to noise because of its high bandwidth in spatial frequencies.

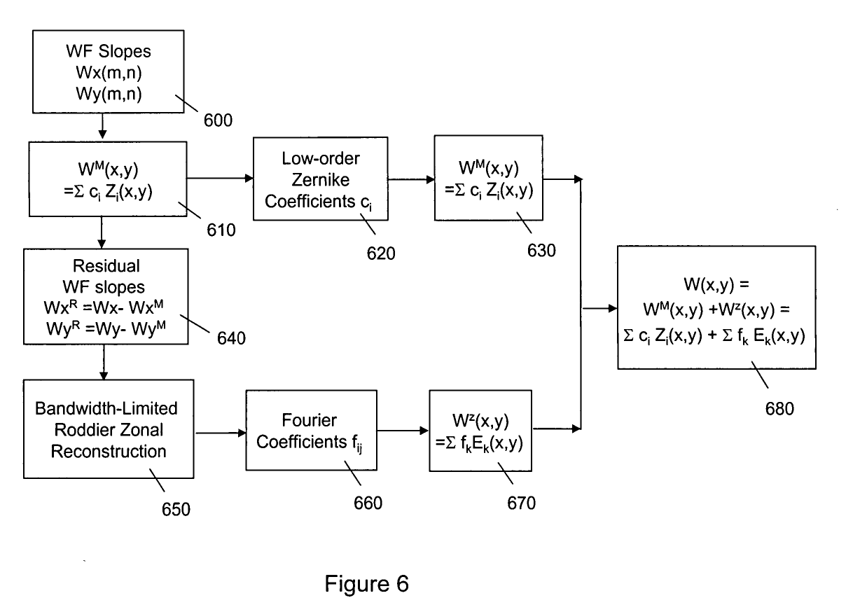

In another embodiment, the invention system and methods provide a mixed modal-zonal wavefront reconstruction that takes advantages of both wavefront reconstruction approaches and at the same time avoids their disadvantages. FIG. 6 shows a flow diagram for a mixed zonal-modal wavefront reconstruction according to the present invention. The wavefront slopes Wx(n,m) and Wy(n,m) in x- and y-directions are obtained in step 600. The wavefront slopes are then fitted with low lower Zernike polynomials that contain most well-know aberrations in step 610 to obtain the Zernike coefficients Ci in step 620. The model portion of the wavefront W^{M}(x,y) is calculated by W^{M}(x,y)=ΣCi Zi(x,y) in step 630.

The residual wavefront slopes Wx^{R} and Wy^{R} are next calculated in step 640. In accordance with the present invention, the residual wavefront slopes is the difference between the measured wavefront slopes and the partial derivatives (Wx^{M} and Wy^{M}) of the model portion of the wavefront W^{M}(x,y) in x- and y-direction, respectively. The residual wavefront data Wx^{R} and Wy^{R} are then fitted with a modified Roddier algorithm for wavefront reconstruction using iterative Fourier transforms in step 650. The Roddier's algorithm (Roddier, 91) uses properties of Fourier series and a filter in the spectral domain and finds the value of the reconstructed wavefront using an iterative method.

In order to filter out unwanted noises in the wavefront slopes, we propose to introduce a low-pass filter in the frequency domain. The bandwidth of the low-pass filter is adjustable in the invention system: low for normal eyes but high for abnormal eyes wherein higher frequency components are needed to properly describe the aberrations.

Another feature of the invention system is that the wavefront data includes the Fourier coefficients in the frequency domain in addition to the spatial data. Fourier coefficients fi are obtained in step 660. W^{Z}(x,y) is calculated in step 670 to approximate the residual wavefront W^{R}(x,y).

The total reconstructed wavefront is finally obtained in step 680 by adding the model portion of the wavefront W^{M}(x,y) represented by Zernike polynomials and the residual portion W^{Z}(x,y) represented by the Fourier series.

The above described mixed modal-zonal wavefront reconstruction in the invention system can improve the accuracy for representing wave aberrations in patients' eyes. First, the lower order Zernike reconstruction extracts all significant aberration modes without being limited by the zonal estimator. Second, an improved Roddier zonal method is used to reconstruct the residual wavefront errors that are local and irregular in nature. Third, an adjustable low-pass filter is applied to remove the high frequency noise in the wavefront data. The frequency bandwidth of the filtering can be adjusted according to the specific eyes. Fourth, wavefront of the eye are represented by Zernike coefficients for conventional aberrations and Fourier coefficients for more irregular wavefront. Finally, the wavefront data at any pupil position can be calculated from Zernike coefficients and Fourier coefficients without the limitations in the conventional interpolation approaches.

A number of embodiments have been described. Nevertheless, it will be understood that various modifications may be made without departing from the spirit and scope of the invention. For example, advantageous results still could be achieved if steps of the disclosed techniques were performed in a different order and/or if components in the disclosed systems were combined in a different manner and/or replaced or supplemented by other components. Accordingly, other embodiments are within the scope of the following claims.

What is claimed is:

1. A method for analyzing wavefront sensing images of a Hartmann-Shack wavefront sensor, comprising:
obtaining a wavefront sensing image of an optical object having at least one optical surface using the Hartmann-Shack wavefront sensor, wherein the wave sensing image comprises an array of focus spots; and
determining at least one average distance between the neighboring focus spots in the wavefront images.

2. The method of claim 1, wherein the optical object is a human eye, and wherein obtaining the wavefront sensing image comprises:
illuminating the retina of the eye with a light beam to produce a compact light source at the retina;
receiving the outgoing wavefront originated from the compact light source at the retina of the patient's eye by an array of lenslets; and
obtaining a wavefront sensing image by an image sensor, wherein the wavefront sensing image includes an array of focus spots each formed by a lenslet in the array of lenslets.

3. The method of claim 2, further comprising:
determining the sphereo-cylindrical corrections for the human eye using the average distance between the neighboring focus spot without finding the locations of the focus spots.

4. The method of claim 2, further comprising:
finding the locations of the focus spots in the wavefront sensing image using the average distance between the neighboring focus spots;
calculating the wavefront slopes from the location of the focus spots; and
determining the wave aberration of the human eye.

5. The method of claim 1, further comprising:
finding the position of a first focus spot around the center of the wavefront sensing image;
determining locations of focus spots adjacent to the first spot using the position of the first focus spot and the average distance between the neighboring focus spots; and
determining the locations of other focus spots using the position of the found spots and the average distance between the neighboring focus spots.

6. The method of claim 1, wherein determining the average distance between the neighboring focus spots comprises:
converting the 2D wavefront image to at least one 1D intensity profile; and
determining the average distance between the neighboring focus spots from the average distance between the neighboring peaks in the 1D intensity profile.

7. The method of claim 6, further comprising
calculating a Fourier spectrum of the 1D intensity profile; and
determining the average spot distance between the neighboring focus spots from the spatial frequencies of spectral peaks in the frequency domain.

8. The method of claim 7, wherein calculating the Fourier spectrum comprises generating at least one Fast Fourier Transform.

9. The method of claim 6, further comprising:
determining the sphereo-cylindrical corrections of the optical object using the average distance between the neighboring focus spot without finding the locations of the focus spots.

10. The method of claim 6, wherein the optical object is a human eye.

11. The method of claim 6, further comprising
finding locations of the focus spots in the wavefront sensing images using the average distance between the focus spots;
calculating the wavefront slopes from the location of the focus spots; and
determining the wave aberration of the optical object.

12. The method of claim 11, wherein the optical object is a human eye.

13. The method of claim 1, wherein determining at least one average distance between the neighboring focus spots comprises:
calculating Fourier transformation of the wavefront image, and
determining the average spot distance between the neighboring focus spots from the spatial frequency of at least one spectral peaks in the frequency domain

14. A method for characterizing the wave aberration of an optical object having at least an optical surface, comprising:
measuring wavefront slopes at an array of sampling points; and
reconstructing the wave aberration of the optical object using the wavefront slopes and a set of Zernike polynomials, wherein the Zernike order is dynamically set to be larger than 10 and less than or equal to the total number of sampling points along one axis in the sample area.

15. The method of claim 14, wherein measuring the wavefront slopes at the array of sampling points uses a Hartmann-Shack sensor.

16. The method of claim 14, wherein the optical object is a human eye.

17. A method for characterizing the wavefront of an optical object having at least an optical surface, comprising:
measuring wavefront slopes at an array of sampling points;
using a modal wavefront estimator to extract a set of wavefront modes from the measured wavefront slopes;
subtracting the set of wavefront modes from the measured wavefront slopes to obtain residual wavefront slopes at the sampling points of the wavefront sensor;
using a zonal wavefront estimator to reconstruct the residual aberrations from the residue wavefront slopes; and
determining the wave aberration of the optical object by combining the set of wavefront modes and the residual aberrations.

18. The method of claim 17, wherein measuring wavefront slopes at an array of sampling points using a Hartmann-Shack sensor.

19. The method of claim 17, wherein the optical object is a human eye.

20. The method of claim 17, wherein the modal wavefront estimator is capable of extracting wavefront modes up to 4^{th} Zernike polynomials.

21. The method of claim 17, wherein the zonal wavefront estimator includes Roddier iterative algorithm that includes a Fourier transformation of the measured wavefront slopes and a spatial filtering the Fourier transformed of the wavefront slopes in the frequency domain.

22. The method of claim 21, further comprising
a low-pass filter in the frequency domain configured to remove the high frequency noise from the residue wavefront slopes.
